(19) United States
(12) Patent Application Publication
(10) Pub. No.: US 2023/0217809 A1
(43) Pub. Date: Jul. 6, 2023

(54) ORGANIC ELECTRONIC ELEMENT, DISPLAY PANEL COMPRISING THE SAME AND DISPLAY DEVICE COMPRISING THE SAME

(71) Applicant: LG DISPLAY CO., LTD., Seoul (KR)

(72) Inventors: Seongsu Jeon, Paju-si (KR); Jicheol Shin, Paju-si (KR); Seonkeun Yoo, Paju-si (KR); SangBeom Kim, Paju-si (KR)

(73) Assignee: LG DISPLAY CO., LTD., Seoul (KR)

(21) Appl. No.: 17/969,372

(22) Filed: Oct. 19, 2022

(30) Foreign Application Priority Data
Dec. 31, 2021 (KR) 10-2021-0193733

(51) Int. Cl. H01L 51/00; C07C 255/52; C09K 11/06

(52) U.S. Cl. CPC H01L 51/0052; C07C 255/52; C09K 11/06; H01L 51/5278

(57) ABSTRACT

An organic electronic element, a display panel and a display device can each include a charge generating layer and a hole injection layer including a p-type dopant with low absorption rate for a blue wavelength band so that they can have excellent light extraction efficiency or luminous efficiency.

### CROSS-REFERENCE TO RELATED APPLICATION

This application claims priority to Korean Patent Application No. 10-2021-0193733, filed in the Republic of Korea on Dec. 31, 2021, which is hereby incorporated by reference in its entirety.

### BACKGROUND

### Field of the Present Disclosure

The present disclosure relates to an organic electronic element, a display panel, and a display device including the organic electronic element.

### Description of the Background Art

In general, an organic light emitting phenomenon refers to the phenomenon of converting electrical energy into light energy by using an organic material. An organic electronic element refers to an electronic element using the organic light emitting phenomenon.

The organic electronic element using the organic light emitting phenomenon can be applied to a display device. Since the portable display device is driven by a battery, which is a limited power source, the organic electronic element used in the portable display device needs excellent light emission efficiency.

In addition, since the image should be displayed properly during use of the electronic device, a long life of the organic electronic element can be also needed.

In order to improve efficiency, lifespan and driving voltage in the organic electronic element, research has been conducted on the organic material included in the organic electronic element and the organic electric device with a multi-stack structure.

### SUMMARY OF THE DISCLOSURE

An organic electronic element including a multi-stack structure combines the lights generated from each light emitting unit and emits the lights to the outside. The intensity and efficiency of the lights generated from the light emitting unit are low while repeatedly passing through the layers of the organic electronic element.

Accordingly, the inventors of the present disclosure have invented an organic electronic element, a display panel, and a display device which include a charge generating layer and a hole injection layer including a p-type dopant with low absorption rate for a blue wavelength band so that they can have excellent light extraction efficiency or luminous efficiency.

The present disclosure is to provide an organic electronic element, a display panel, and a display device that can have high light extraction efficiency or luminous efficiency.

In an aspect of the present disclosure, an organic electronic element includes a first electrode; a second electrode; an organic layer positioned between the first electrode and the second electrode, and the organic layer includes a first stack including a first light emitting layer, a second stack including a second light emitting layer; a charge generating layer positioned between the first stack and the second stack; and a hole injection layer positioned between the first electrode and the first light emitting layer.

In the organic electronic element of the present disclosure, any one of the charge generating layer and the hole injection layer includes a first compound represented by Chemical Formula 1 in an amount of from 1 wt. % to 30 wt. %.

In an aspect of the present disclosure, a display panel includes a subpixel including the organic electronic element.

In an aspect of the present disclosure, a display device includes the display panel, and a driving circuit for driving the display panel.

According to various aspects of the present disclosure, in an organic electronic element, a display panel, and a display device, a charge generating layer and a hole injection layer include a p-type dopant with low absorption rate for a blue wavelength band, thereby having excellent light extraction efficiency or luminous efficiency.

### BRIEF DESCRIPTION OF THE DRAWINGS

The above and other objects, features, and advantages of the disclosure will be more clearly understood from the following detailed description, taken in conjunction with the accompanying drawings, in which:

FIG. 1 is a system configuration diagram of a display device according to aspects of the present disclosure;

FIG. 2 is a view illustrating a sub-pixel circuit of a display panel according to aspects of the present disclosure;

FIG. 3 is a cross-sectional view schematically illustrating an organic electronic element for describing a light extraction path of the two stack top emission type;

FIG. 4 is a graph illustrating the absorbance for blue light spectrum according to comparative example;

FIG. 5 is a schematic cross-sectional view of an organic electronic element according to aspects of the present disclosure;

FIG. 6 is a schematic cross-sectional view of an organic electronic element according to other aspects of the present disclosure; and

FIG. 7 is a graph illustrating the comparison of the absorbance for blue light spectrum according to aspects of the present disclosure and comparative example.

### DETAILED DESCRIPTION OF THE EMBODIMENTS

In the following description of examples or embodiments of the disclosure, reference will be made to the accompanying drawings in which it is shown by way of illustration specific examples or embodiments that can be implemented, and in which the same reference numerals and signs can be used to designate the same or like components even when they are shown in different accompanying drawings from one another. Further, in the following description of examples or embodiments of the disclosure, detailed descriptions of well-known functions and components incorporated herein will be omitted when it is determined that the description can make the subject matter in some embodiments of the disclosure rather unclear. The terms such as “including”, “having”, “containing”, “constituting” “make up of”, and “formed of” used herein are generally intended to allow other components to be added unless the terms are used with the term “only”. As used herein, singular forms are intended to include plural forms unless the context clearly indicates otherwise.

Terms, such as “first”, “second”, “A”, “B”, “(A)”, or “(B)” can be used herein to describe elements of the disclosure. Each of these terms is not used to define essence, order, sequence, or number of elements etc., but is used merely to distinguish the corresponding element from other elements.

When it is mentioned that a first element “is connected or coupled to”, “contacts or overlaps” etc. a second element, it should be interpreted that, not only can the first element “be directly connected or coupled to” or “directly contact or overlap” the second element, but a third element can also be “interposed” between the first and second elements, or the first and second elements can “be connected or coupled to”, “contact or overlap”, etc. each other via a fourth element. Here, the second element can be included in at least one of two or more elements that “are connected or coupled to”, “contact or overlap”, etc. each other.

When time relative terms, such as “after,” “subsequent to,” “next,” “before,” and the like, are used to describe processes or operations of elements or configurations, or flows or steps in operating, processing, manufacturing methods, these terms can be used to describe non-consecutive or non-sequential processes or operations unless the term “directly” or “immediately” is used together.

In addition, when any dimensions, relative sizes etc. are mentioned, it should be considered that numerical values for an elements or features, or corresponding information (e.g., level, range, etc.) include a tolerance or error range that can be caused by various factors (e.g., process factors, internal or external impact, noise, etc.) even when a relevant description is not specified. Further, the term “can” fully encompasses all the meanings of the term “can”.

Hereinafter, various aspects of the present disclosure are described in detail with reference to the accompanying drawings.

As used herein, the term “halo” or “halogen” includes fluorine (F), chlorine (Cl), bromine (Br), and iodine (I), and the like, unless otherwise specified.

As used herein, the term “alkyl” or “alkyl group” can mean a radical of a saturated aliphatic functional group having 1 to 60 carbon atoms linked by a single bond and including a straight chain alkyl group, branched chain alkyl group, cycloalkyl (alicyclic) group, alkyl-substituted cycloalkyl group, or cycloalkyl-substituted alkyl group, unless otherwise specified.

As used herein, the term “haloalkyl group” or “halogen alkyl group” can mean a halogen-substituted alkyl group unless otherwise specified.

As used herein, the term “alkenyl” or “alkynyl” can have a double bond or a triple bond, respectively, and can include a straight or branched chain group and can have 2 to 60 carbon atoms unless otherwise specified.

As used herein, the term “cycloalkyl” can refer to an alkyl forming a ring having 3 to 60 carbon atoms, unless otherwise specified.

As used herein, the term “alkoxy group” or “alkyloxy group” refers to an alkyl group to which an oxygen radical is bonded, and can have 1 to 60 carbon atoms unless otherwise specified.

As used herein, the term “alkenoxyl group”, “alkenoxy group”, “alkenyloxyl group”, or “alkenyloxy group” refers to an alkenyl group to which an oxygen radical is attached, and can have 2 to 60 carbon atoms unless otherwise specified.

As used herein, the terms “aryl group” and “arylene group” each can have 6 to 60 carbon atoms unless otherwise specified, but are not limited thereto. In the disclosure, the aryl group or the arylene group can include a monocyclic type, a ring assembly, a fused polycyclic system, a spiro compound, and the like. For example, the aryl group includes, but is not limited to, phenyl, biphenyl, naphthyl, anthryl, indenyl, phenanthryl, triphenylenyl, pyrenyl, peryleneyl, chrysenyl, naphthacenyl, or fluoranthenyl. The naphthyl can include 1-naphthyl and 2-naphthyl, and the anthryl can include 1-anthryl, 2-anthryl and 9-anthryl.

In the disclosure, the term “fluorenyl group” or “fluorenylene group” can refer to a monovalent or divalent functional group, respectively, of fluorene, unless otherwise specified. The “fluorenyl group” or “fluorenylene group” can mean a substituted fluorenyl group or a substituted fluorenylene group. “Substituted fluorenyl group” or “substituted fluorenylene group” can refer to a monovalent or divalent functional group of substituted fluorene. “Substituted fluorene” can mean that at least one of the following substituents R, R′, R″ and R′″ is a functional group other than hydrogen. It can include a case where R and R′ are bonded to each other to form a spiro compound together with the carbon to which they are bonded.

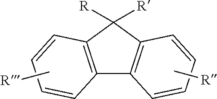

As used herein, the term “spiro compound” has a ‘spiro union’, and the spiro union means a union formed as two rings share only one atom. In this case, the atom shared by the two rings can be referred to as a ‘spiro atom’. The compounds are defined as ‘monospiro-’, ‘dispiro-’ or ‘trispiro-’ depending on the number of spiro atoms in one compound.

As used herein, the term “heterocyclic group” can include not only an aromatic ring, such as a “heteroaryl group” or “heteroarylene group” but also a non-aromatic ring and, unless otherwise specified, means a ring with 2 to 60 carbon atoms and one or more heteroatoms, but is not limited thereto.

As used herein, the term “heteroatom” refers to N, O, S, P or Si unless otherwise specified. The “heterocyclic group” can mean a monocyclic group containing a heteroatom, a ring assembly, a fused polycyclic system, or a spiro compound.

The “heterocyclic group” can include a ring containing SO₂ instead of carbon forming the ring. For example, the “heterocyclic group” can include the following compounds.

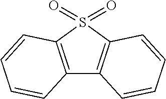 [SMILES: O=S1(=O)c2ccccc2-c2ccccc21]

As used herein, the term “ring” can include monocycles and polycycles, can include hydrocarbon rings as well as heterocycles containing at least one heteroatom, or can include aromatic and non-aromatic rings.

As used herein, the term “polycycle” can include ring assemblies, fused polycyclic systems, and spiro compounds, can include aromatic as well as non-aromatic compounds, or can include heterocycles containing at least one heteroatom as well as hydrocarbon rings.

As used herein, the term “aliphatic ring group” refers to a cyclic hydrocarbon other than the aromatic hydrocarbon, can include a monocyclic type, a ring assembly, a fused polycyclic system, and a spiro compound and, unless otherwise specified, can mean a ring having 3 to 60 carbon atoms. For example, a fusion of benzene, which is an aromatic ring, and cyclohexane, which is a non-aromatic ring, also corresponds to an aliphatic ring.

As used herein, the term “alkyl silyl group” can refer to a monovalent substituent in which three alkyl groups are bonded to a Si atom.

As used herein, the term “aryl silyl group” can refer to a monovalent substituent in which three aryl groups are bonded to a Si atom.

As used herein, the term “alkyl aryl silyl group” can refer to a monovalent substituent in which one alkyl group and two aryl groups are bonded to a Si atom or two alkyl groups and one aryl group are bonded to the Si atom.

As used herein, the term “ring assembly” means that two or more ring systems (single or fused ring systems) are directly connected to each other through single or double bonds. For example, in the case of an aryl group, a biphenyl group or a terphenyl group can be a ring assembly but is not limited thereto.

As used herein, the term “fused polycyclic system” refers to a type of fused rings sharing at least two atoms. For example, in the case of an aryl group, a naphthalenyl group, a phenanthrenyl group, or a fluorenyl group can be a fused polycyclic system, but is not limited thereto.

When prefixes are named successively, it can mean that the substituents are listed in the order specified first. For example, an arylalkoxy group can mean an alkoxy group substituted with an aryl group, an alkoxycarbonyl group can mean a carbonyl group substituted with an alkoxy group, and an arylcarbonylalkenyl group can mean an alkenyl group substituted with an arylcarbonyl group. The arylcarbonyl group can be a carbonyl group substituted with an aryl group.

Unless otherwise explicitly stated, in the term “substituted” or “unsubstituted” as used herein, “substituted” can mean being substituted with one or more substituents selected from the group consisting of halogen, an amino group, a nitrile group, a nitro group, a C₁-C₂₀ alkyl group, a C₁-C₂₀ alkoxy group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₂₀ aryl group, a C₈-C₂₀ arylalkenyl group, a silane group, a boron group, a germanium group, and a C₂-C₂₀ heterocyclic group including at least one heteroatom selected from the group consisting of O, N, S, Si and P, but is not limited to the substituents.

In the disclosure, the ‘functional group names’ corresponding to the aryl group, arylene group, and heterocyclic group provided as examples of the symbols and their substituents can be described with ‘the names of the functional groups reflecting the valence’, but can also be described with ‘the names of the parent compounds.’ For example, in the case of ‘phenanthrene’, which is a type of aryl group, its name can be specified with its group identified, such as ‘phenanthryl (group)’ for the monovalent group, and ‘phenanthrylene (group)’ as the divalent group, but can also be specified as ‘phenanthrene’, which is the name of the parent compound, regardless of the valence. Similarly, pyrimidine can be specified as ‘pyrimidine’ regardless of the valence or can also be specified as pyrimidinyl (group) for the monovalence and as pyrimidylene (group) for the divalence. Therefore, in the disclosure, when the type of the substituent is specified with the name of the parent compound, it can mean an n-valent ‘group’ formed by detachment of the hydrogen atom bonded to a carbon atom and/or a heteroatom of the parent compound.

Further, unless explicitly stated, the formulas used in the disclosure can be applied in the same manner as the definition of the substituent by the following formulas.

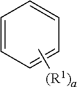

When a is 0, it means that the substituent R¹ does not exist, meaning that hydrogen is bonded to each of the carbon atoms forming the benzene ring. In this case, the chemical formula or chemical compound can be specified without expressing the hydrogen bonded to the carbon. Further, when a is 1, one substituent R¹ is bonded to any one of the carbon atoms forming the benzene ring, and when a is 2 or 3, it can be bonded as follows. When a is an integer of 4 to 6, it is bonded to the carbon of the benzene ring in a similar manner, and when a is an integer of 2 or more, R¹ can be identical or different.

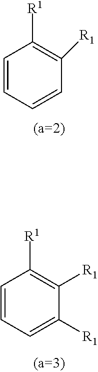

In the disclosure, when substituents are bonded to each other to form a ring, it can mean that adjacent groups are bonded to each other to form a monocycle or fused polycycle, and the monocycle or fused polycycle can include heterocycles containing at least one heteroatom as well as hydrocarbon rings and can include aromatic and non-aromatic rings.

In the disclosure, an organic light emitting element can mean a component(s) between the anode and the cathode or an organic light emitting diode including an anode, a cathode, and component(s) positioned therebetween.

In some cases, in the disclosure, an organic light emitting element can mean an organic light emitting diode and a panel including the same, or an electronic device including the panel and circuitry. The electronic device can include, e.g., a display device, a lighting device, a solar cell, a portable or mobile terminal (e.g., a smart phone, a tablet, a PDA, an electronic dictionary, or PMP), a navigation terminal, a game device, various TVs, and various computer monitors but, without limited thereto, can include any type of device including the component(s).

Hereinafter, various aspects of the present disclosure are described in detail with reference to the accompanying drawings.

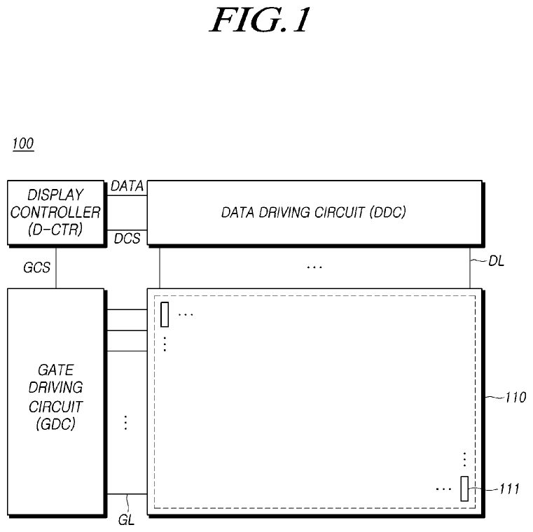

FIG. 1 is a system configuration diagram of a display device according to aspects of the present disclosure.

Referring to FIG. 1, the display device 100 in according to aspects of the present disclosure includes a display panel 110 in which a plurality of data lines DL and a plurality of gate lines GL are arranged and a plurality of sub-pixels 111 defined by the plurality of data lines DL and the plurality of gate lines GL is arranged, a data driving circuit DDC (or a data driver) for driving the plurality of data lines DL, a gate driving circuit GDC (or a gate driver) for driving the plurality of gate lines GL, a controller D-CTR controlling the data driving circuit DDC and the gate driving circuit GDC, and the like.

The controller D-CTR controls operations of the data driving circuit DDC and the gate driving circuit GDC by supplying respective control signals (DCS, GCS) to the data driving circuit DDC and the gate driving circuit GDC.

The controller D-CTR starts the scan of pixels according to timings processed in each frame, converts image data inputted from other devices or other image providing sources to be adapted to a data signal form used in the data driving circuit DDC and then outputs image data DATA resulted from the converting, and causes the data to be loaded into the pixels at a pre-configured time according to the scan.

The controller D-CTR can be implemented as a separate component from the data driving circuit DDC or can be integrated with data driving circuit DDC so the controller D-CTR can be implemented as an integrated circuit.

The data driving circuit DDC drives the plurality of data lines DL by providing data voltages corresponding to image data DATA received from the controller D-CTR to the data lines DL. Here, the data driving circuit DDC is sometimes referred to as a source driving circuit or a source driver.

The data driving circuit DDC can include at least one source driver integrated circuit SDIC to be implemented.

Each source driver integrated circuit SDIC can include a shift register, a latch circuit, a digital to analog converter DAC, an output buffer, and/or the like.

In some instances, each source driver integrated circuit SDIC can further include one or more analog to digital converters ADC.

The gate driving circuit GDC sequentially drives a plurality of gate lines GL by sequentially providing scan signals to the plurality of gate lines GL. Here, the gate driving circuit GDC is sometimes referred to as a scan driving circuit or a scan driver.

The gate driving circuit GDC can include at least one gate driver integrated circuit GDIC to be implemented.

Each gate driver integrated circuit GDIC can include a shift register, a level shifter, and/or the like.

Each gate driver integrated circuit GDIC can be connected to a bonding pad of the display panel 110 in a tape automated bonding (TAB) type or a chip on glass (COG) type, or be directly disposed on the display panel 110 as being implemented in a gate in panel (GIP) type. In some instances, the gate driver integrated circuit GDIC can be disposed to be integrated with the display panel 110. Further, each gate driver integrated circuit GDIC can be implemented in a chip on film (COF) type in which the gate driver integrated circuit GDIC is mounted on a film connected with the display panel 110.

According to the controlling of the controller D-CTR, the gate driving circuit GDC sequentially provides scan signals of an on-voltage or an off-voltage to the plurality of gate lines GL.

When a specific gate line is asserted by a scan signal from the gate driving circuit GDC, the data driving circuit DDC converts image data DATA received from the controller D-CTR into analog data voltages and provides the obtained analog data voltages to the plurality of data lines DL.

The data driving circuit DDC can be located on, but not limited to, only one side (e.g., an upper side or a lower side) of the display panel 110, or in some instances, be located on, but not limited to, two sides (e.g., the upper side and the lower side) of the display panel 110 according to driving schemes, panel design schemes, or the like.

The gate driving circuit GDC can be located on, but not limited to, only one side (e.g., a left side or a right side) of the panel 110, or in some instances, be located on, but not limited to, two sides (e.g., the left side and the right side) of the display panel 110 according to driving schemes, panel design schemes, or the like.

The display device 100 according to aspects of the present disclosure can be one of various types of display devices, such as, a liquid crystal display device, an organic light emitting display device, a plasma display device, or the like.

In case the display device 100 according to aspects of the present disclosure is an organic light emitting display device, each sub-pixel 111 arranged in the display panel 110 can include circuit components, such as an organic light emitting diode (OLED), which is a self-emissive element, a driving transistor for driving the organic light emitting diode OLED, and the like.

Types of circuit elements and the number of the circuit elements included in each subpixel 111 can be different depending on types of the panel (e.g., an LCD panel, an OLED panel, etc.), provided functions, design schemes/features, or the like.

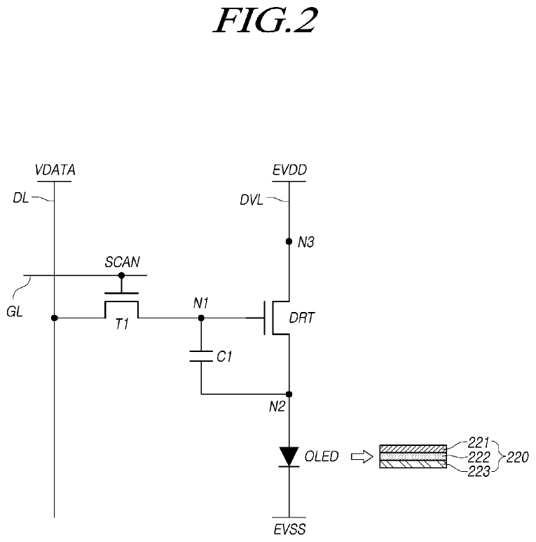

FIG. 2 is a view illustrating a subpixel circuit of a display panel according to aspects.

Referring to FIG. 2, each subpixel 111 can include an organic light emitting diode OLED and a driving transistor DRT for driving the organic light emitting diode OLED as basic circuit components.

Referring to FIG. 2, each sub-pixel 111 can further include a first transistor T1 allowing a data voltage VDATA to be applied to a first node N1 corresponding to a gate node of the driving transistor DRT, and a storage capacitor C1 for remaining a data voltage VDATA corresponding to an image signal voltage or a voltage corresponding to this during one frame time.

The organic light emitting diode OLED can include a first electrode 221 (an anode electrode or a cathode electrode), a light emitting layer 222, a second electrode 223 (the cathode electrode or the anode electrode), and the like.

In one aspect, a low-level voltage EVSS can be applied to the second electrode 223 of the organic light emitting diode OLED.

The driving transistor DRT causes the organic light emitting diode OLED to be driven by providing a driving current to the organic light emitting diode OLED.

The driving transistor DRT includes a first node N1, a second node N2 and a third node N3.

The first node N1 of the driving transistor DRT can be a node corresponding to the gate node thereof, and can be electrically connected to a source node or a drain node of the first transistor T1.

The second node N2 of the driving transistor DRT can be electrically connected to the first electrode 221 of the organic light emitting diode OLED and can be a source node or a drain node.

The third node N3 of the driving transistor DRT can be the drain node or the source node as a node to which a driving voltage EVDD is applied, and can be electrically connected to a driving voltage line DVL used to supply a driving voltage EVDD.

The first transistor T1 can be electrically connected between a data line DL and the first node N1 of the driving transistor DRT and can be controlled by a scan signal SCAN that is provided through a gate line and applied to the gate node of the first transistor T1.

The storage capacitor C1 can be electrically connected between the first node N1 and the second node N2 of the driving transistor DRT.

The storage capacitor C1 is an external capacitor intentionally designed to be located outside of the driving transistor DRT, not an internal storage, such as a parasitic capacitor (e.g., a Cgs, a Cgd) that presents between the first node N1 and the second node N2 of the driving transistor DRT.

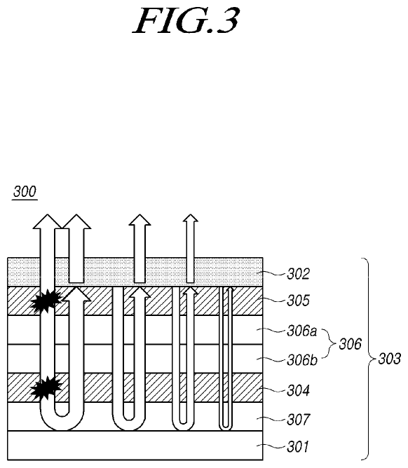

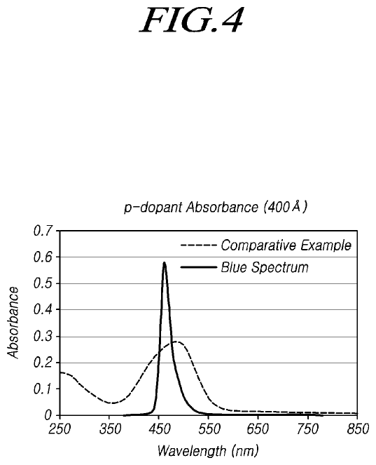

FIG. 3 is a cross-sectional view schematically illustrating an organic electronic element for describing a light extraction path of the two stack top emission type. FIG. 4 is a graph illustrating the absorbance for blue light spectrum according to comparative example used as a conventional p-type dopant.

Referring to FIG. 3, the organic electric device 300 can include a plurality of light emitting layers 304 and 305. Among the light emitted from the second light emitting layer 305 located at the top of the light emitting layers, light that passes through the second electrode 302 and is directly emitted upwards and light that is reflected by the first electrode 301 and then is emitted upwards can be divided.

As described above, the micro-cavity effect is that light is repeatedly reflected between the first electrode 301 and the second electrode 302. That is, light is repeatedly reflected in the cavity between the first electrode 301 and the second electrode 302 by the fine resonance effect, thereby increasing the luminous efficiency.

However, in the case of a strong cavity structure using an anode electrode having a high reflectance as the first electrode 301 and a cathode electrode having a semitransparent reflective film as the second electrode 302, many reflections occur between the anode and the cathode, and light is emitted after repeatedly passing through the hole injection layer 307 and the charge generating layer 306.

Referring to FIG. 4, a conventional p-type dopant has a high absorbance in a wavelength band of 350 nm˜600 nm, and particularly shows a significantly high absorbance in a wavelength band of about 450 nm˜500 nm including a wavelength band of blue light.

Referring to FIGS. 3 and 4, light generated from the light emitting layers 304 and 305, particularly blue light repeatedly passes through the hole injection layer 307 and the charge generating layer 306, and the blue light is absorbed by the p-type dopant included in the charge generating layer 306 and the intensity of the blue light is weakened by the absorption rate of the p-type dopant.

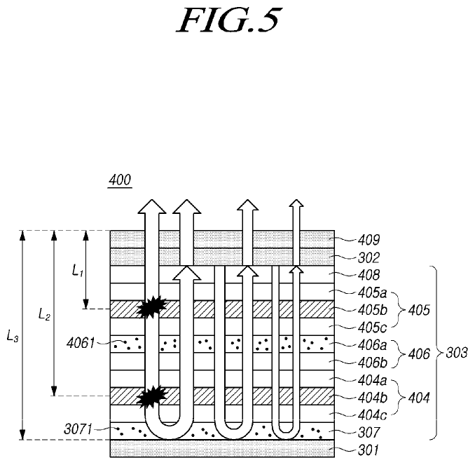

FIG. 5 is a schematic cross-sectional view of an organic electronic element according to aspects of the present disclosure.

Referring to FIG. 5, an organic electronic element 400 according to aspects of the present disclosure can include a first electrode 301, a second electrode 302, and an organic material layer 303 positioned between the first electrode 301 and the second electrode 302.

For example, the first electrode 301 can be an anode electrode, and the second electrode 302 can be a cathode electrode.

For example, the first electrode 301 can be a transparent electrode having a high transmittance, and the second electrode 302 can be a semitransparent reflective electrode. In another example, the first electrode 301 can be a semitransparent reflective electrode, and the second electrode 302 can be a transparent electrode having a high transmittance.

The organic material layer 303 can be a layer positioned between the first electrode 301 and the second electrode 302 and including an organic material and can be composed of a plurality of layers.

The organic material layer 303 can include a first stack 404, a second stack 405, and a charge generating layer 406 positioned between the first stack 404 and the second stack 405.

The organic material layer 303 can include a hole injection layer 307 positioned between the first electrode 301 and a first light emitting layer 404b.

Any one or both layers of the charge generating layer 406 and the hole injection layer 307 can include a first compounds 3071 and 4061 represented by Chemical Formula 1 in an amount of from about 1 wt. % to 30 wt. %, for example about 2 wt. % to 25 wt. %. When the first compounds 3071 and 4061 satisfies the above amount, the absorbance for a wavelength band of blue light in the charge generating layer and the hole injection layer can be reduced, thereby improving light extraction efficiency and luminous efficiency.

The organic electronic element 400 can be a tandem-type organic electronic element including a plurality of stacks each including a light emitting layer. The plurality of light emitting layers can be formed of the same material or different materials. The plurality of light emitting layers can emit light of the same color or a different color. For example, all of the plurality of light emitting layers can be blue light emitting layers, some can be blue light emitting layers, and some can be red, green or yellow light emitting layers.

The first stack 404 can include a first light emitting layer 404b. The first light emitting layer 404b can include, e.g., a host compound and a dopant.

The first light emitting layer 404b can be disposed to have a distance within a range of from 1800 Å to 2400 Å from a surface of the second electrode 302, and can be disposed to have a distance within a range of from 2600 Å to 3200 Å.

The first stack 404 can further include a first hole transport layer 404c and a first electron transport layer 404a.

The first hole transport layer 404c can be positioned between the first light emitting layer 404b and the first electrode 301 or and the second electrode 302 as the anode electrode. The first electron transport layer 404a can be positioned between the first light emitting layer 404b and the second electrode 302 or and the first electrode 301 as the cathode electrode.

For example, when the first electrode 301 is the anode electrode and the second electrode 302 is the cathode electrode, the first hole transport layer 404c can be positioned on the first electrode 301, the first light emitting layer 404b can be positioned on the first hole transport layer 404c, and the first electron transport layer 404a can be positioned on the first light emitting layer 404b.

The second stack 405 can include a second light emitting layer 405b. The second light emitting layer 405b can include, e.g., a host compound and a dopant.

The second light emitting layer 405b can be disposed to have a distance within a range of from 200 Å to 800 Å from a surface of the second electrode 302, and can be disposed to have a distance within a range of from 1000 Å to 1600 Å.

The second stack 405 can further include a second hole transport layer 405c and a second electron transport layer 405a.

The second hole transport layer 405c can be positioned between the second light emitting layer 405b and the first electrode 301 or and the second electrode 302 as the anode electrode. The second electron transport layer 405a can be positioned between the second light emitting layer 405b and the second electrode 302 or and the first electrode 301 as the cathode electrode. For example, when the first electrode 301 is the anode electrode and the second electrode 302 is the cathode electrode, the second hole transport layer 405c can be positioned on the first electrode 301, the second light emitting layer 405b can be positioned on the second hole transport layer 405c, and the second electron transport layer 405a can be positioned on the second light emitting layer 405b.

As the first stack 404, and the second stack 405 can be configured as described above, the holes and electrons transferred from the first electrode 301 and the second electrode 302 meet at the first light emitting layer 404b, and the second light emitting layer 405b, emitting light.

The charge generating layer 406 can be formed between the plurality of light emitting layers to smoothly distribute charges, thereby increasing the current efficiency of the light emitting layer. Accordingly, the charge generating layer 406 can be positioned between the first stack 404 including the first light emitting layer 404b and the second stack 405 including the second light emitting layer 405b.

The charge generating layer 406 can include a p-type charge generating layer and an n-type charge generating layer to smoothly distribute charges. For example, a first layer 406a can be a p-type charge generating layer, and a second layer 406b can be an n-type charge generating layer. When the first electrode 301 is the anode electrode, and the second electrode 302 is the cathode electrode, the p-type charge generating layer can be positioned on the side of the cathode electrode, and the n-type charge generating layer can be positioned on the side of the anode electrode. For example, the first layer 406a can be positioned between the second layer 406b and the second electrode 302 as the cathode electrode.

The first layer 406a can include a first compound 4061. The first layer 406a can be a layer including the first compound 4061, can be composed of a plurality of layers each including one or more of the first compound 4061. Although FIG. 5 illustrates the first layer 406a including one layer, aspects of the present disclosure are not limited thereto.

The first compound 4061 included in the first layer 406a can be a p-type dopant, the first layer 406a can include the first compound 4061 in an amount of from about 3 wt. % to 30 wt. %, for example about 10 wt. % to 25 wt. %. When the first compound 4061 satisfies the above amount, the absorbance for a wavelength band of blue light in the charge generating layer can be reduced, thereby improving light extraction efficiency and luminous efficiency.

Although FIG. 5 illustrates a tandem-type organic electronic element including two stacks, aspects of the present disclosure are not limited thereto but can include tandem-type organic electronic elements including two or more stacks.

When the organic electronic element 400 can include an additional stack, an additional charge generating layer can be positioned between the additional stack and the first stack 404 or second stack 405 adjacent thereto.

The organic electronic element 400 can include a hole injection layer 307.

The hole injection layer 307 can be positioned between the first electrode 301 as an anode electrode and the first light emitting layer 404b. For example, the hole injection layer 307 can be positioned between the first electrode 301 as an anode electrode and the first hole transport layer 404c.

The hole injection layer 307 can include a first compound 3071. The first compound 3071 included in the hole injection layer 307 can be a p-type dopant, the hole injection layer 307 can include the first compound 3071 in an amount of from about 1 wt. % to 15 wt. %, for example about 2 wt. % to 8 wt. %. When the first compound 3071 satisfies the above amount, the absorbance for a wavelength band of blue light in the hole injection layer can be reduced, thereby improving light extraction efficiency and luminous efficiency.

The first compounds 3071 and 4061 included in the charge generating layer 406 and the hole injection layer 307 can be the same as each other or can be different from each other.

The first compounds 3071 and 4061 included in the charge generating layer 406 and the hole injection layer 307 are compounds represented by Chemical Formula 1, and can be the same as or different from each other.

The organic electronic element 400 can include an electron injection layer 308.

For example, the electron injection layer 308 can be positioned between the second electrode 302 as a cathode electrode and the second electron transport layer 405a.

The organic electronic element 400 can include a capping layer 309.

For example, the capping layer 309 can be positioned on the second electrode 302 as a cathode electrode.

In another example, each of the first stack 404 and the second stack 405 can further include one or more of a hole injection layer and an electron injection layer. Within each stack, the hole injection layer can be located between the light emitting layer and the anode electrode, and an electron injection layer can be located between the light emitting layer and the cathode electrode.

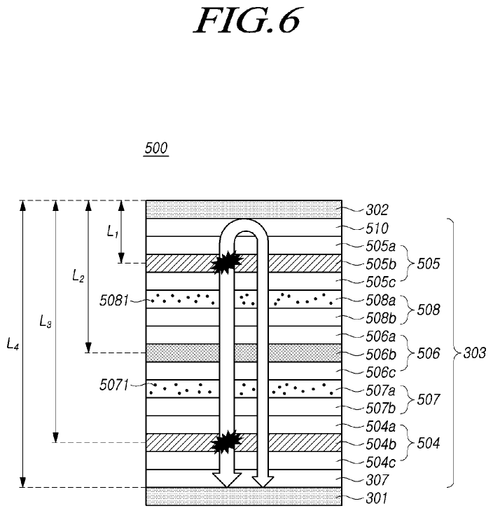

FIG. 6 is a schematic cross-sectional view of an organic electronic element according to other aspects of the present disclosure.

Referring to FIG. 6, an organic electronic element 500 according to aspects of the present disclosure can include a first charge generating layer 507, a third stack 506 and a second charge generating layer 508 between the first stack and the second stack in the organic electronic element 400 described above, a description of the same configuration will be omitted.

The first charge generating layer 507 can be positioned between the first stack 504 including the first light emitting layer 504b and the third stack 506 including a third light emitting layer 506b.

The first charge generating layer 507 can include a p-type charge generating layer and an n-type charge generating layer to smoothly distribute charges. For example, a third layer 507a can be a p-type charge generating layer, and a fourth layer 507b can be an n-type charge generating layer. When the first electrode 301 is the anode electrode, and the second electrode 302 is the cathode electrode, the p-type charge generating layer can be positioned on the side of the cathode electrode, and the n-type charge generating layer can be positioned on the side of the anode electrode. For example, the third layer 507a can be positioned between the fourth layer 507b and the second electrode 302 as the cathode electrode.

The third layer 507a can include a first compound 5071. The third layer 507a can be a layer including the first compound 5071, can be composed of a plurality of layers each including one or more of the first compound 5071. Although FIG. 6 illustrates the third layer 507a including one layer, aspects of the present disclosure are not limited thereto.

The first compound 5071 included in the third layer 507a can be a p-type dopant, the third layer 507a can include the first compound 5071 in an amount of from about 5 wt. % to 30 wt. %, for example about 10 wt. % to 25 wt. %. When the first compound 5071 satisfies the above amount, the absorbance for a wavelength band of blue light in the charge generating layer can be reduced, thereby improving light extraction efficiency and luminous efficiency.

The third stack 506 can include a third light emitting layer 506b. The third light emitting layer 506b can include, e.g., a host compound and a dopant.

The third stack 506 can further include a third hole transport layer 506c and a third electron transport layer 506a.

The third hole transport layer 506c can be positioned between the third light emitting layer 506b and the first electrode 301 or and the second electrode 302 as the anode electrode. The third electron transport layer 506a can be positioned between the third light emitting layer 506b and the second electrode 302 or and the first electrode 301 as the cathode electrode

For example, when the first electrode 301 is the anode electrode and the second electrode 302 is the cathode electrode, the third hole transport layer 506c can be positioned on the first electrode 301, the third light emitting layer 506b can be positioned on the third hole transport layer 506c, and the third electron transport layer 506a can be positioned on the third light emitting layer 506b.

The second charge generating layer 508 can be positioned between the second stack 505 including the second light emitting layer 505b and the third stack 506 including a third light emitting layer 506b.

The second charge generating layer 508 can include a p-type charge generating layer and an n-type charge generating layer to smoothly distribute charges. For example, a fifth layer 508a can be a p-type charge generating layer, and a sixth layer 508b can be an n-type charge generating layer. When the first electrode 301 is the anode electrode, and the second electrode 302 is the cathode electrode, the p-type charge generating layer can be positioned on the side of the cathode electrode, and the n-type charge generating layer can be positioned on the side of the anode electrode. For example, the fifth layer 508a can be positioned between the sixth layer 508b and the second electrode 302 as the cathode electrode.

The fifth layer 508a can include a first compound 5081. The fifth layer 508a can be a layer including the first compound 5081, can be composed of a plurality of layers each including one or more of the first compound 5081. Although FIG. 6 illustrates the fifth layer 508a including one layer, aspects of the present disclosure are not limited thereto.

The first compound 5081 included in the fifth layer 508a can be a p-type dopant, the fifth layer 508a can include the first compound 5081 in an amount of from about 5 wt. % to 30 wt. %, for example about 10 wt. % to 25 wt. %. When the first compound 5081 satisfies the above amount, the absorbance for a wavelength band of blue light in the charge generating layer can be reduced, thereby improving light extraction efficiency and luminous efficiency.

A compound represented by Chemical Formula 1 described above is described below.

The compound can be represented by Chemical Formula 1 as follows.

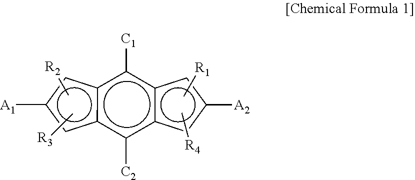

In Chemical Formula 1, A₁ and A₂ can be each independently selected from the group consisting of a hydrogen; a halogen; a cyano group; a nitro group; a C₆-C₆₀ aryl group; a fluorenyl group; a C₂-C₆₀ heterocyclic group containing at least one heteroatom selected from the group consisting of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group.

C₁ and C₂ can be each independently a halogen; or a cyano group.

R₁ to R₄ can be each independently selected from the group consisting of a hydrogen; a halogen; a cyano group; a nitro group; a C₆-C₆₀ aryl group; a fluorenyl group; a C₂-C₆₀ heterocyclic group containing at least one heteroatom selected from the group consisting of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group, and at least two of R₁ to R₄ can be a cyano group.

When one or more of A₁, A₂ and R₁ to R₄ are aryl groups, the aryl groups can be a C₆-C₃₀ aryl group, a C₆-C₂₀ aryl group, or a C₆-C₁₂ aryl group.

When one or more of A₁, A₂ and R₁ to R₄ are heterocyclic groups, the heterocyclic groups can be a C₂-C₃₀ heterocyclic group, a C₂-C₂₀ heterocyclic group, or a C₂-C₁₂ heterocyclic group.

The aryl group, the fluorenyl group, the heterocyclic group, the fused ring group, the alkyl group, the alkenyl group, the alkynyl group, the alkoxy group and the aryloxy group can be each further substituted with one or more substituents selected from the group consisting of a nitro group; a cyano group; a halogen; an amino group; a C₁-C₂₀ alkoxyl group; a C₁-C₂₀ alkylthio group; a C₁-C₂₀ alkyl group; a C₂-C₂₀ alkenyl group; a C₂-C₂₀ alkynyl group; a C₆-C₂₀ aryl group; a fluorenyl group; a C₂-C₂₀ heterocyclic group; a C₃-C₂₀ cycloalkyl group; a C₇-C₂₀ arylalkyl group; and a C₈-C₂₀ arylalkenyl group.

One or more of the hydrogen atoms contained in the compound represented by Chemical Formula 1 can be substituted with deuterium or tritium.

In an organic electronic element, a charge generating layer and a hole injection layer include the compound represented by Chemical Formula 1 as a p-type dopant with low absorption rate for a blue wavelength band, thereby having excellent light extraction efficiency or luminous efficiency.

The compound represented by Chemical Formula 1 can be represented by either Chemical Formula 1-1 or the Chemical Formula 1-2 below.

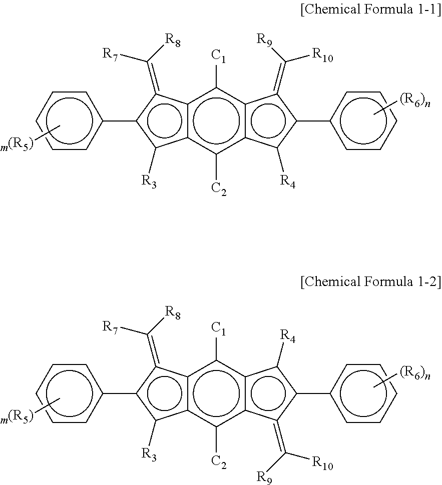

In Chemical Formula 1-1 and Chemical Formula 1-2, C₁, C₂, R₃, and R₄ can be the same as defined in Chemical Formula 1.

m and n can be each independently an integer from 0 to 5.

R₅ to R₁₀ can be each independently selected from the group consisting of a hydrogen; a halogen; a cyano group; a C₁-C₂₀ alkyl group; and a C₁-C₂₀ alkoxy group.

The alkyl group and the alkoxy group can be each further substituted with one or more substituents selected from the group consisting of a halogen; and a cyano group.

One or more of the hydrogen atoms contained in the compound represented by Chemical Formula 1-1 or Chemical Formula 1-2 can be substituted with deuterium or tritium.

The compound represented by Chemical Formula 1 is one or more of the following compounds below.

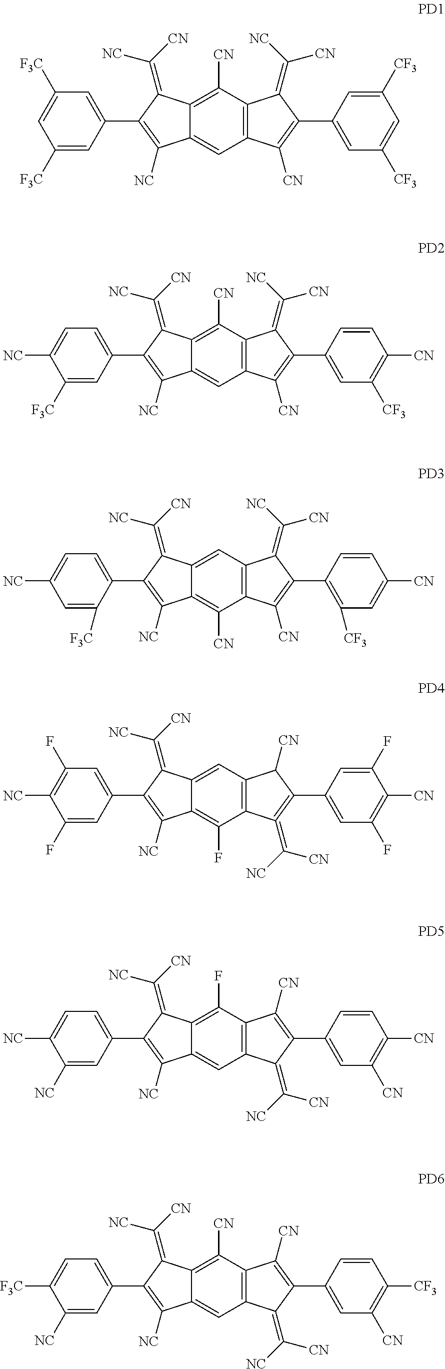

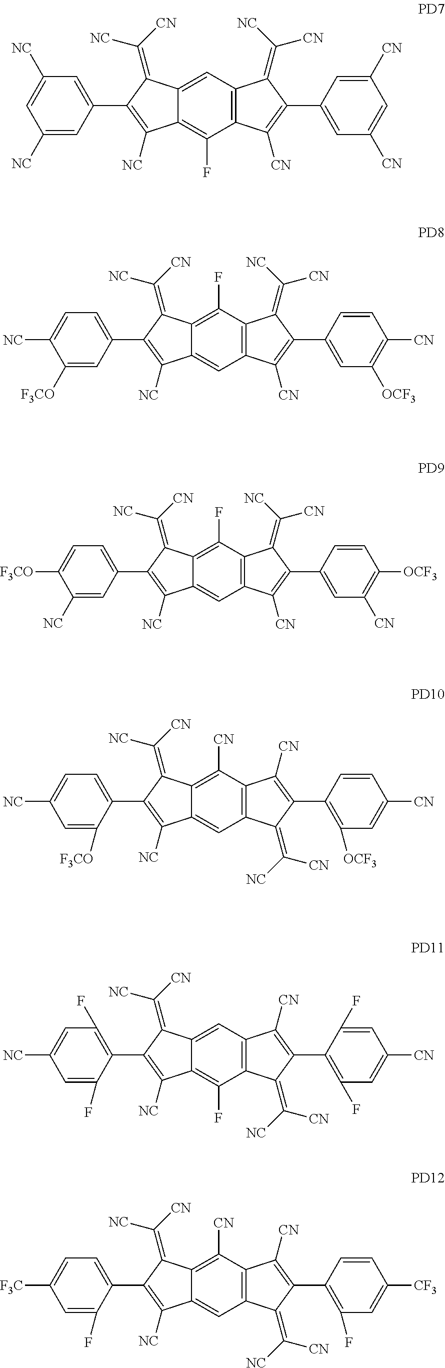

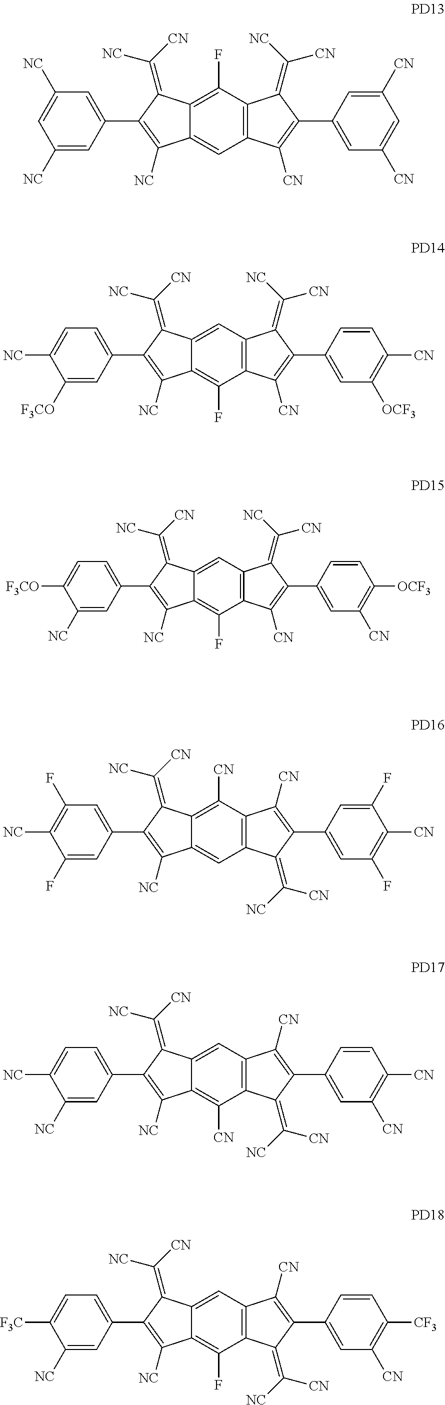

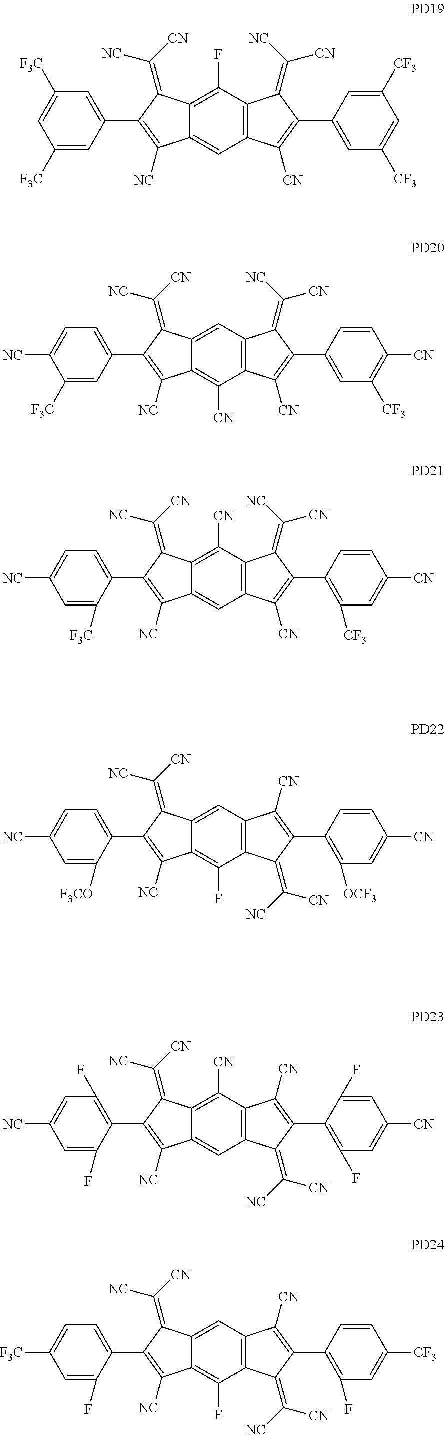

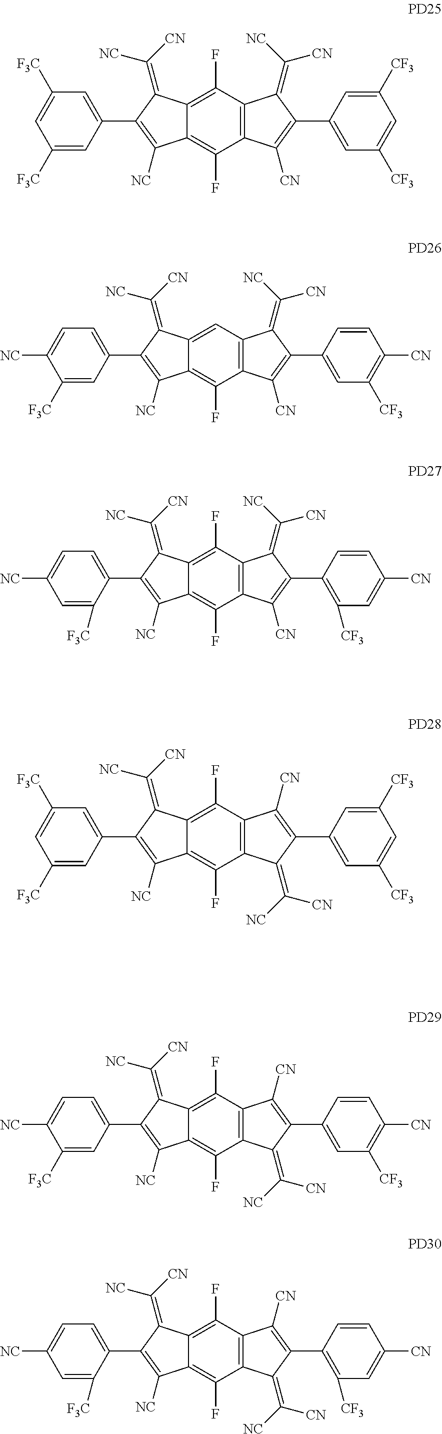

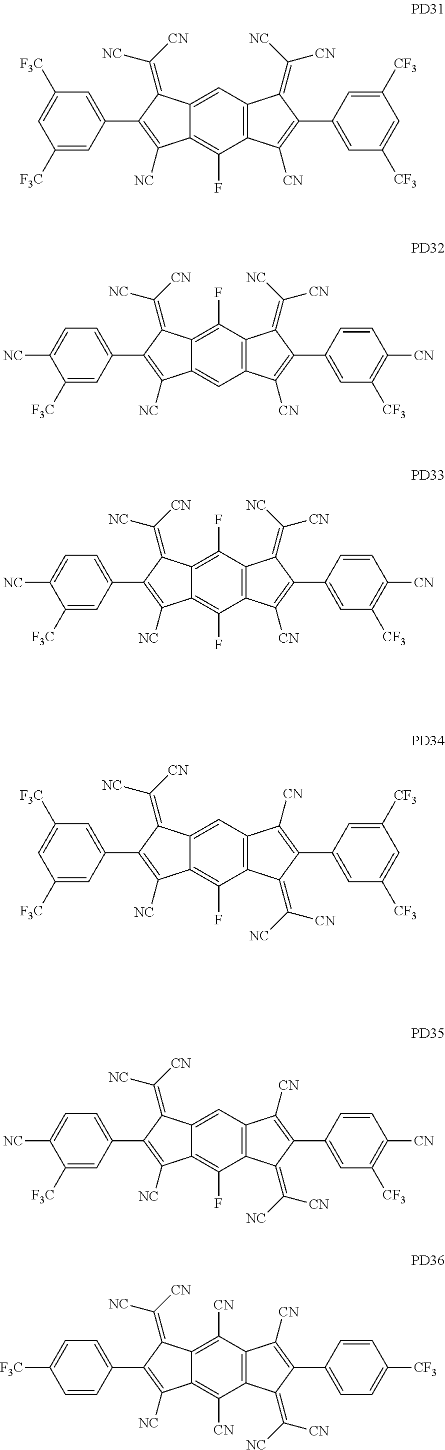

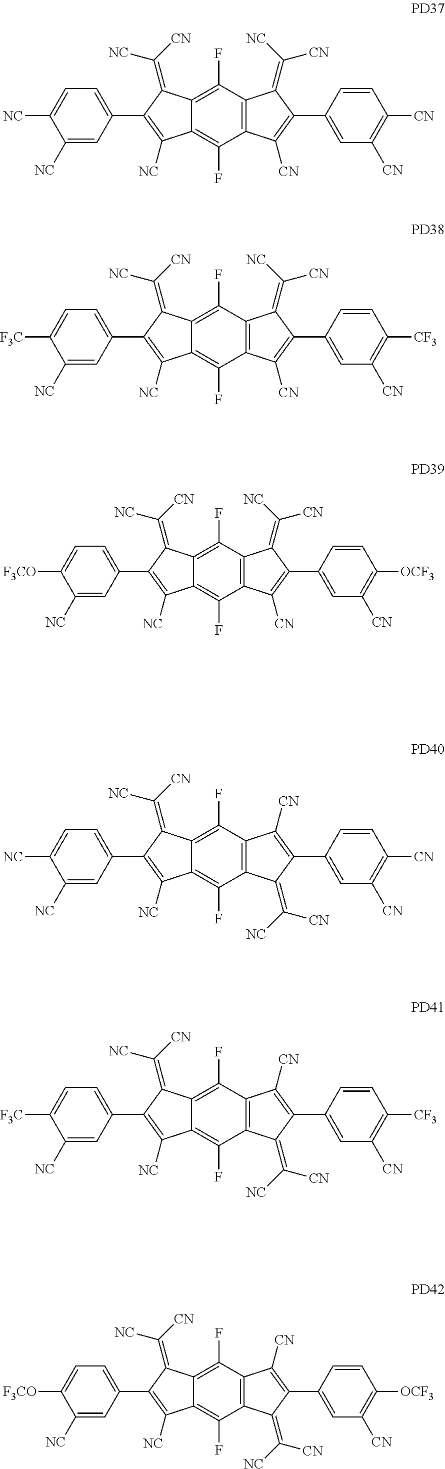

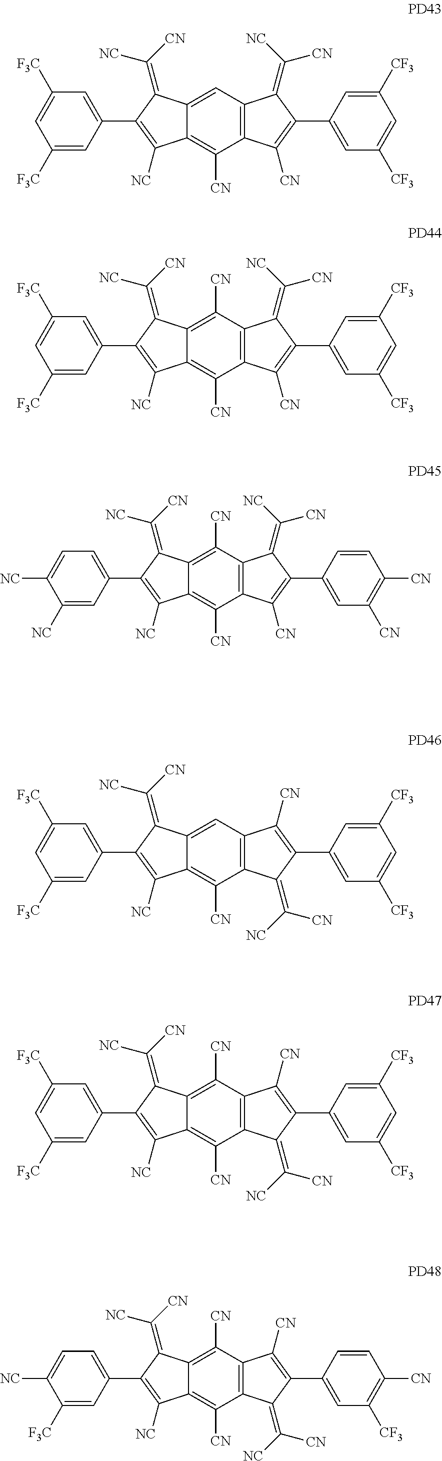

One or more of the hydrogen atoms included in PD1 to PD48 can be substituted with deuterium or tritium.

In aspects of the present disclosure can provide a display panel 110.

The display panel 110 includes a sub-pixel 111 including the above-described organic electronic element 220.

In the display panel 110 according to aspects of the present disclosure, since the organic electronic element 220 is the same as the organic electronic element 400, 500 according to the above-described aspects, a description thereof will be omitted.

In addition, since the display panel 110 and the sub-pixel 111 according to aspects of the present disclosure have been described above, a description thereof will be omitted.

In aspects of the present disclosure can provide a display device 100.

The display device 100 includes the above-described display panel 110 and a driving circuit for driving the above-described display panel 110.

In the display device 100 according to aspects of the present disclosure, the display panel 110 is the same as the display panel 110 according to the above-described aspects, so a description thereof will be omitted.

In addition, since the driving circuit for driving the display panel according to aspects of the present disclosure has been described above, a description thereof will be omitted.

An example of manufacturing an organic electronic element according to aspects of the present disclosure are described below in detail with reference to embodiments thereof, but embodiments of the present disclosure are not limited to the following embodiments.

Manufacturing Evaluation of Organic Electronic Element

1. Efficiency Evaluation According to p-Type Dopant

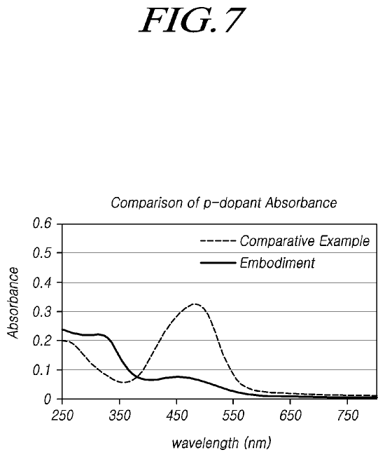

As shown in Table 1 below, in the organic electronic element shown in FIG. 5, which is an aspect of the present disclosure, a hole injection layer (HIL) and a p-type charge generating layer were formed to manufacture a blue organic electronic element having a two stack top emission type. The absorbance and efficiency were evaluated at 460 nm, the blue light wavelength, and FIG. 7 is a graph illustrating the comparison of the absorbance for blue light spectrum according to aspects of the present disclosure and comparative example.

The compounds used in the manufacturing evaluation of organic electronic element are as follows.

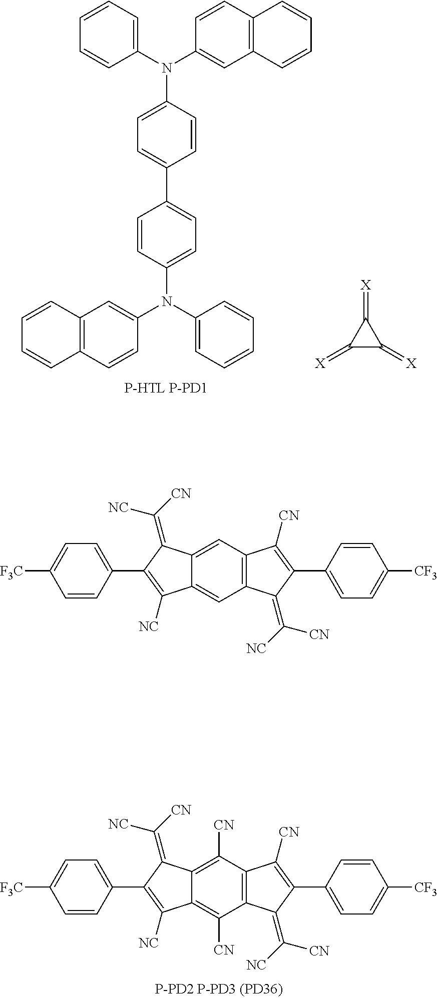

TABLE 1

| | HIL | | p-CGL | | | |
|---|---|---|---|---|---|---|
| | P- | P- | P- | P- | Absorbance | |
| | Host | dopant | Host | dopant | (460 nm) | EQE (%) |
| comparative | P- | P-DO1 | P- | P-DO1 | 100% | 100% |
| example 1 | HTL | | HTL | | | |
| comparative | P- | P-PD2 | P- | P-DO2 | 71% | 103% |
| example 2 | HTL | | HTL | | | |
| embodiment | P- | P-DO3 | P- | P-DO3 | 34% | 111% |
| 1 | HTL | | HTL | | | |

Referring to Table 1 and FIG. 7, when the compound of the present disclosure is used as a p-type dopant of the hole injection layer and the p-type charge generating layer, the absorbance is reduced by 34% compared to comparative example 1 of the prior art, and the efficiency (EQE) is improved by 11%. The compound of comparative example 2 does not have a cyano group in the phenyl located in the middle of the core, but the compound according to an aspect of the present disclosure reduces the absorbance and improves the efficiency by introducing a cyano group.

2. Efficiency Evaluation According to the Concentration of p-Type Dopant

As shown in Table 2 below, in the organic electronic element shown in FIG. 5, which is an aspect of the present disclosure, a hole injection layer (HIL) and a p-type charge generating layer were formed to manufacture a blue organic electronic element having a two stack top emission type. And, voltage difference and efficiency according to the concentration of p-type dopant were evaluated.

TABLE 2

| | HIL | | | p-CGL | | | | |
|---|---|---|---|---|---|---|---|---|
| | P-Host | P-dopant | | P-Host | P-dopant | | ΔV | EQE (%) |
| embodiment 2 | P- | P- | 2% | P- | P- | 14% | 0 | 100% |
| | HTL | DO3 | | HTL | DO1 | | | |
| embodiment 3 | P- | P- | 4% | P- | P- | 14% | −0.04 | 102% |
| | DO3 | DO3 | | HTL | DO1 | | | |
| embodiment 4 | P- | P- | 6% | P- | P- | 14% | −0.05 | 102% |
| | DO3 | DO3 | | HTL | DO1 | | | |
| embodiment 5 | P- | P- | 8% | P- | P- | 14% | −0.07 | 99% |
| | DO3 | DO3 | | HTL | DO1 | | | |
| embodiment 6 | P- | P- | 4% | P- | P- | 10% | 0 | 100% |
| | HTL | DO1 | | HTL | DO3 | | | |
| embodiment 7 | P- | P- | 4% | P- | P- | 15% | −0.1 | 104% |
| | HTL | DO1 | | HTL | DO3 | | | |
| embodiment 8 | P- | P- | 4% | P- | P- | 20% | −0.12 | 101% |
| | HTL | DO1 | | HTL | DO3 | | | |
| embodiment 9 | P- | P- | 4% | P- | P- | 25% | −0.13 | 96% |
| | HTL | DO1 | | HTL | DO3 | | | |

Referring to Table 2, as the concentration of p-type dopant increased in the hole injection layer and the p-type charge generating layer, the driving voltage was lowered or the efficiency (EQE) was improved.

Referring to embodiment 2˜embodiment 5, as the concentration of p-type dopant in the hole injection layer was varied from 2 wt. % to 8 wt. %, the driving voltage was lowered by up to 0.07V, the efficiency (EQE) was improved by up to 102%.

Referring to embodiment 6˜embodiment 9, as the concentration of p-type dopant in the p-type charge generating layer was varied from 10 wt. % to 25 wt. %, the driving voltage was lowered by up to 0.13V, the efficiency (EQE) was improved by up to 104%.

3. Efficiency Evaluation According to the Concentration of p-Type Dopant

As shown in Table 3 below, in the organic electronic element shown in FIG. 6, which is an aspect of the present disclosure, a first p-type charge generating layer and a second p-type charge generating layer were formed to manufacture a blue organic electronic element having a three stack bottom emission type. And, voltage difference and efficiency according to the concentration of p-type dopant were evaluated.

TABLE 3

| | First p-CGL | | | Second p-CGL | | | | |
|---|---|---|---|---|---|---|---|---|
| | P-Host | P-dopant | | P-Host | P-dopant | | ΔV | EQE (%) |
| embodiment 10 | P- | P- | 10% | P- | P- | 10% | 0 | 100% |
| | HTL | DO3 | | HTL | DO3 | | | |
| embodiment 11 | P- | P- | 15% | P- | P- | 15% | −0.09 | 104% |
| | HTL | DO3 | | HTL | DO3 | | | |
| embodiment 12 | P- | P- | 20% | P- | P- | 20% | −0.11 | 102% |
| | HTL | DO3 | | HTL | DO3 | | | |
| embodiment 13 | P- | P- | 25% | P- | P- | 25% | −0.13 | 99% |
| | HTL | DO3 | | HTL | DO3 | | | |

Referring to Table 3, as the concentration of p-type dopant increased in the first p-type charge generating layer and the second p-type charge generating layer, the driving voltage was lowered or the efficiency (EQE) was improved.

Referring to embodiment 10˜embodiment 13, as the concentration of p-type dopant in the first p-type charge generating layer and the second p-type charge generating layer was varied from 10 wt. % to 25 wt. %, the driving voltage was lowered by up to 0.13V, the efficiency (EQE) was improved by up to 104%.

According to various aspects of the present disclosure, in an organic electronic element, a display panel, and a display device, a charge generating layer and a hole injection layer include a p-type dopant with low absorption rate for a blue wavelength band, thereby having excellent light extraction efficiency or luminous efficiency.

The above description has been presented to enable any person skilled in the art to make and use the technical idea of the present disclosure, and has been provided in the context of a particular application and its requirements. Various modifications, additions and substitutions to the described embodiments will be readily apparent to those skilled in the art, and the general principles defined herein can be applied to other embodiments and applications without departing from the spirit and scope of the present disclosure. The above description and the accompanying drawings provide an example of the technical idea of the present disclosure for illustrative purposes only. That is, the disclosed embodiments are intended to illustrate the scope of the technical idea of the present disclosure.

Thus, the scope of the present disclosure is not limited to the embodiments shown, but is to be accorded the widest scope consistent with the claims. The scope of protection of the present disclosure should be construed based on the following claims, and all technical ideas within the scope of equivalents thereof should be construed as being comprised within the scope of the present disclosure.

What is claimed is:

1. An organic electronic element comprising:
a first electrode;
a second electrode;
an organic layer positioned between the first electrode and the second electrode,
wherein the organic layer comprises:
a first stack comprising a first light emitting layer;
a second stack comprising a second light emitting layer;
a charge generating layer positioned between the first stack and the second stack; and
a hole injection layer positioned between the first electrode and the first light emitting layer, and
any one of the charge generating layer and the hole injection layer comprises a first compound represented by the following Chemical Formula 1 in an amount of from 1 wt. % to 30 wt. %: 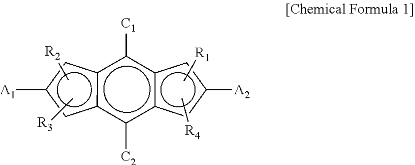
wherein in Chemical Formula 1,
A₁ and A₂ are each independently selected from the group consisting of a hydrogen; a halogen; a cyano group; a nitro group; a C₆-C₆₀ aryl group; a fluorenyl group; a C₂-C₆₀ heterocyclic group containing at least one heteroatom selected from the group consisting of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group,
C₁ and C₂ are each independently a halogen; or a cyano group,
R₁ to R₄ are each independently selected from the group consisting of a hydrogen; a halogen; a cyano group; a nitro group; a C₆-C₆₀ aryl group; a fluorenyl group; a C₂-C₆₀ heterocyclic group containing at least one heteroatom selected from the group consisting of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group,
wherein at least two of R₁ to R₄ are a cyano group, and
the aryl group, the fluorenyl group, the heterocyclic group, the fused ring group, the alkyl group, the alkenyl group, the alkynyl group, the alkoxy group and the aryloxy group can be each further substituted with one or more substituents selected from the group consisting of a nitro group; a cyano group; a halogen; an amino group; a C₁-C₂₀ alkoxyl group; a C₁-C₂₀ alkylthio group; a C₁-C₂₀ alkyl group; a C₂-C₂₀ alkenyl group; a C₂-C₂₀ alkynyl group; a C₆-C₂₀ aryl group; a fluorenyl group; a C₂-C₂₀ heterocyclic group; a C₃-C₂₀ cycloalkyl group; a C₇-C₂₀ arylalkyl group; and a C₈-C₂₀ arylalkenyl group.

2. The organic electronic element according to claim 1, wherein the first compound is represented by either Chemical Formula 1-1 or Chemical Formula 1-2 below: 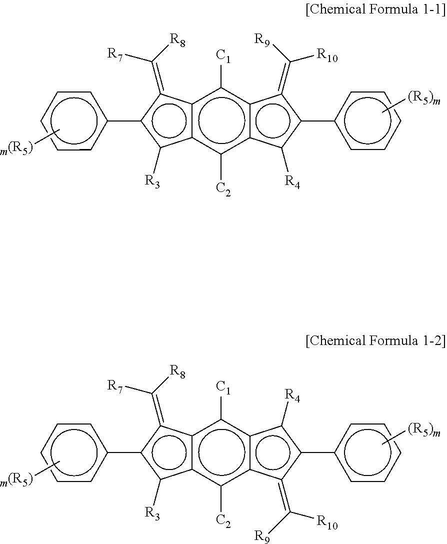
wherein in Chemical Formula 1-1 and Chemical Formula 1-2,
C₁, C₂, R₃, and R₄ are the same as defined in Chemical Formula 1,
m and n are each independently an integer from 0 to 5,
R₅ to R₁₀ are each independently selected from the group consisting of a hydrogen; a halogen; a cyano group; a C₁-C₂₀ alkyl group; and a C₁-C₂₀ alkoxy group, and
the alkyl group and the alkoxy group are each further substituted with one or more substituents selected from the group consisting of a halogen; and a cyano group.

3. The organic electronic element according to claim 1, wherein the first compound is one or more of compounds below:

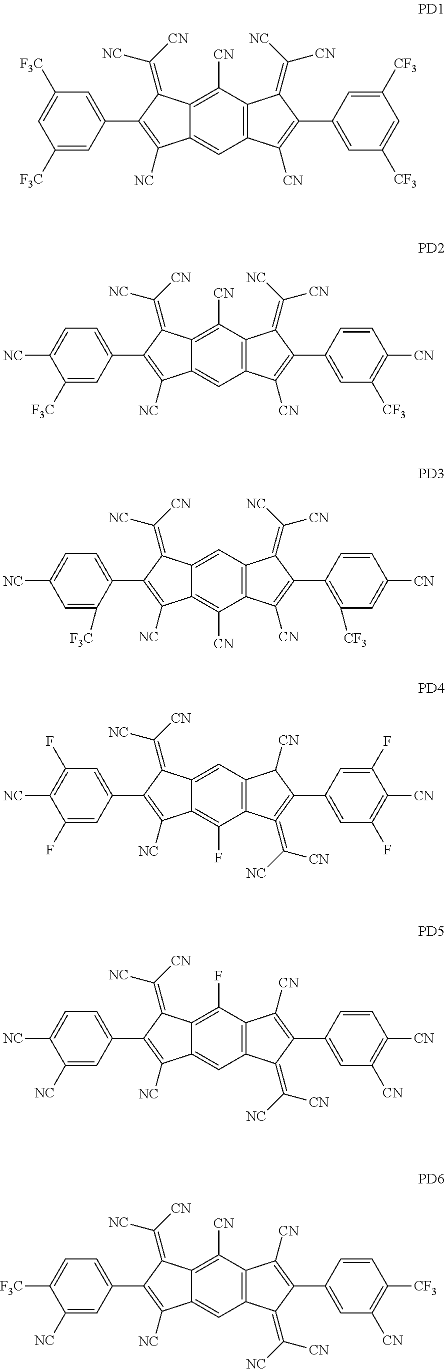

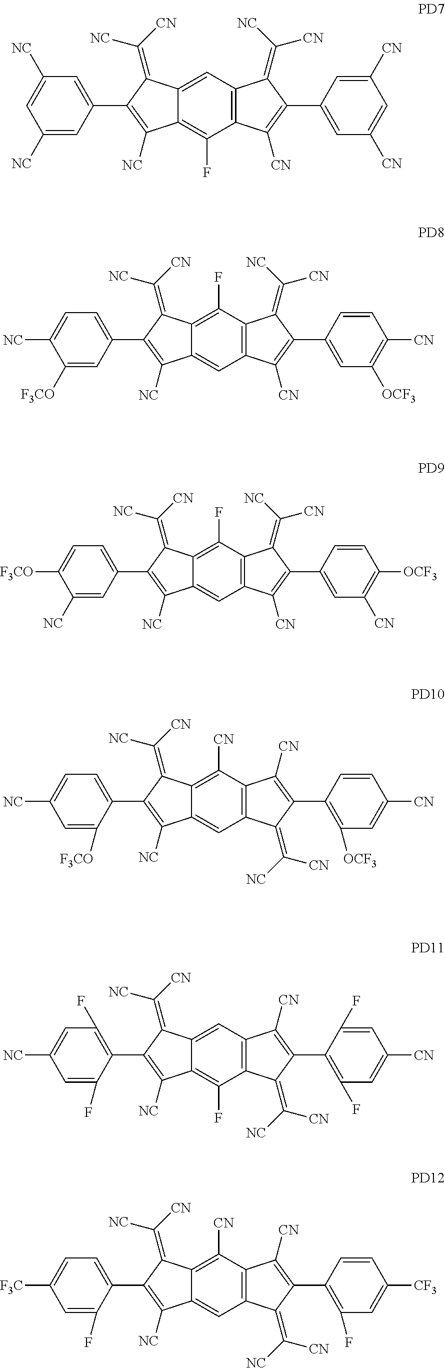

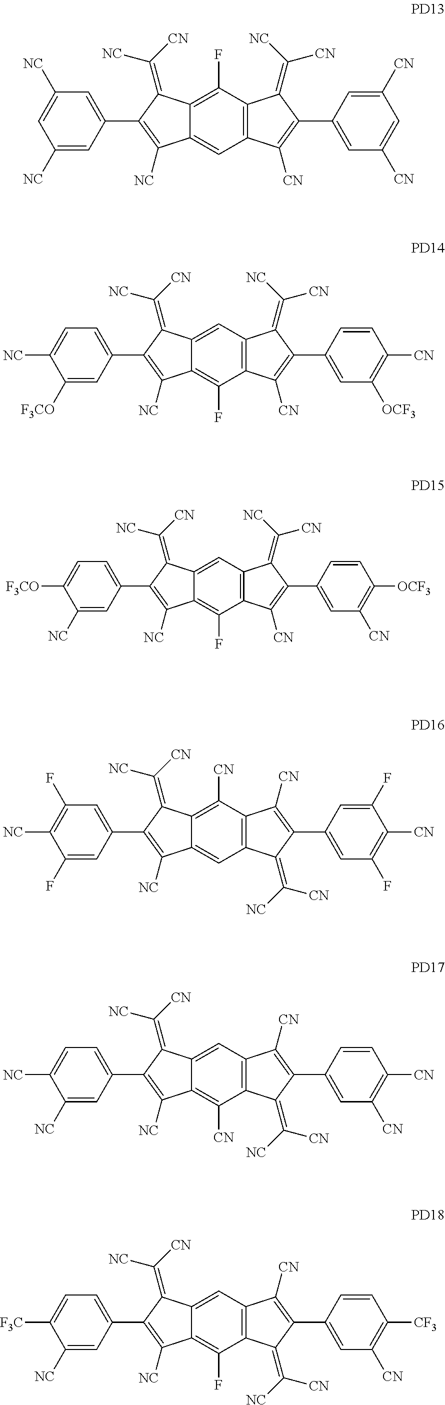

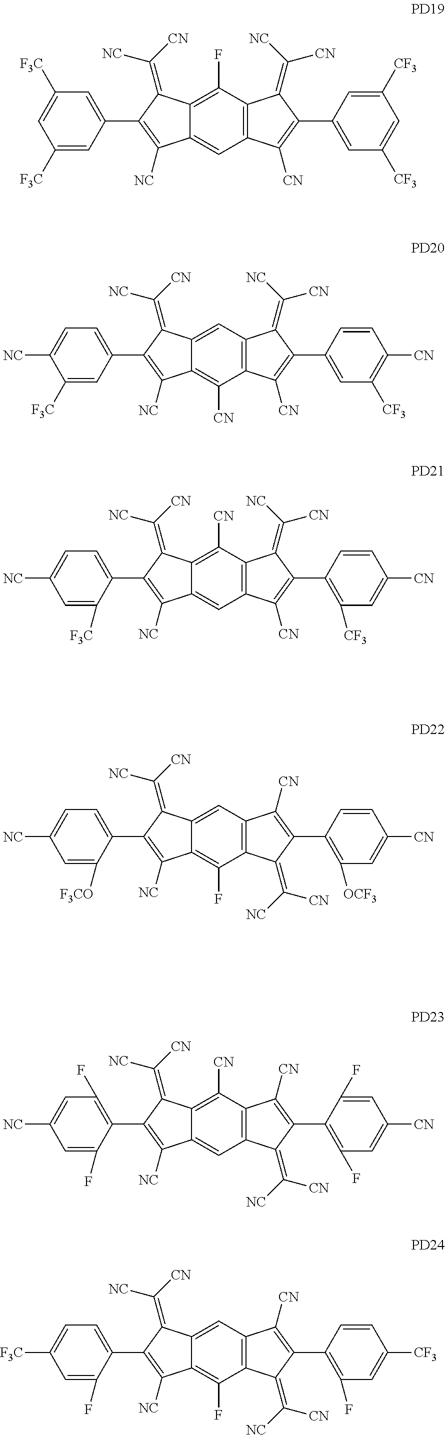

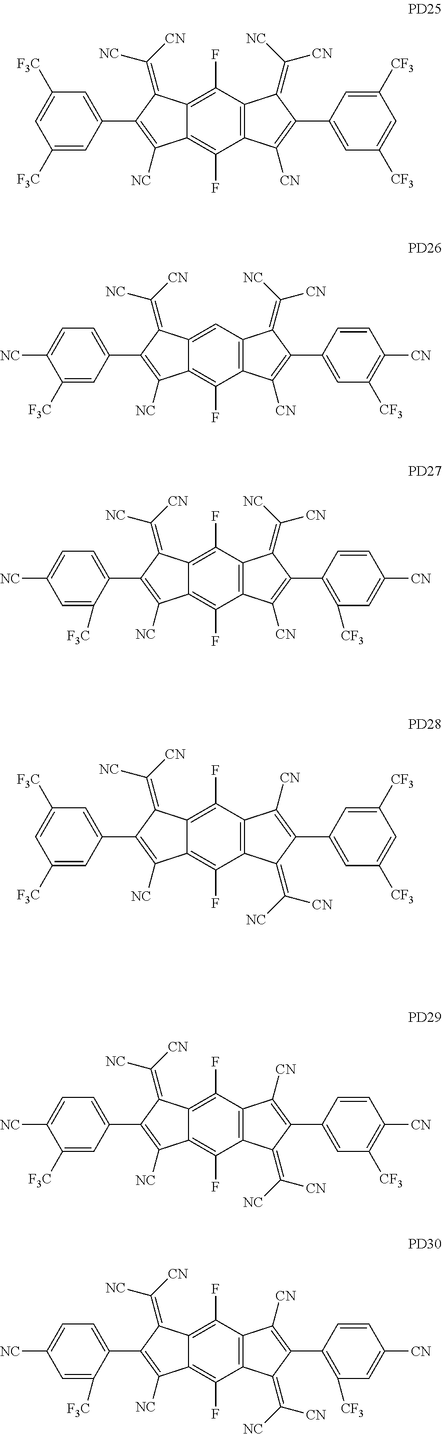

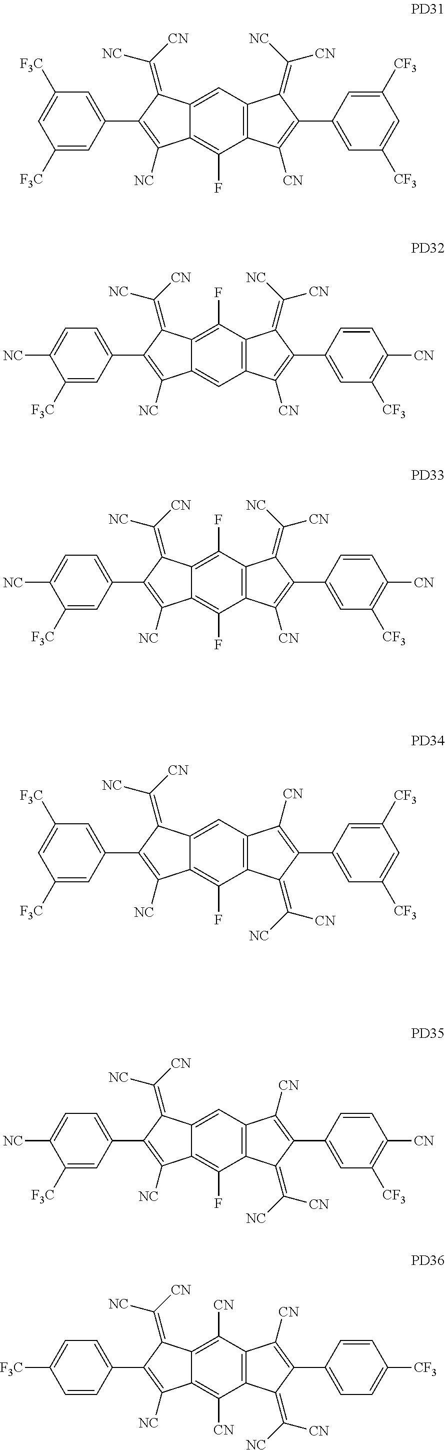

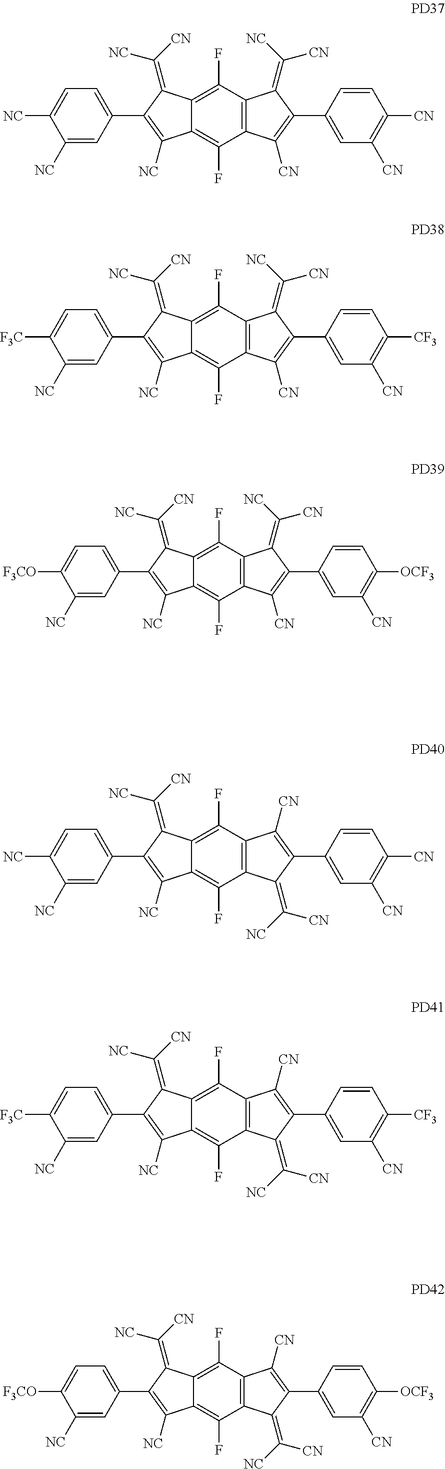

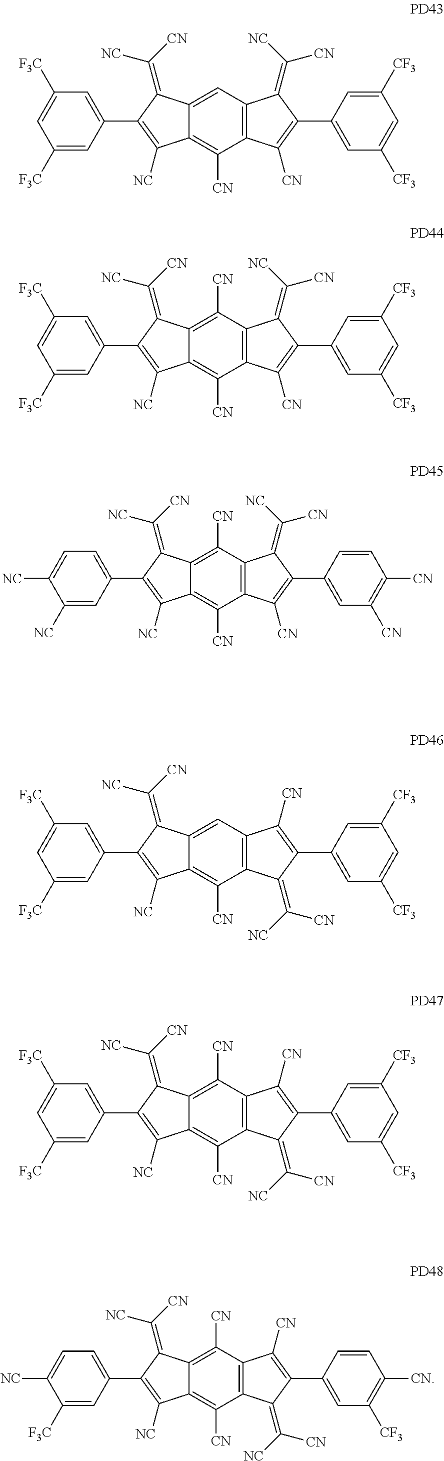

4. The organic electronic element according to claim 1, wherein the charge generating layer comprises a first layer and a second layer, and the first layer comprises the first compound.

5. The organic electronic element according to claim 4, wherein the first layer is a p-type charge generating layer, and the second layer is an n-type charge generating layer, and the first compound is a p-type dopant.

6. The organic electronic element according to claim 4, wherein the first layer comprises the first compound in an amount of from 5 wt. % to 30 wt. %.

7. The organic electronic element according to claim 1, wherein the hole injection layer comprises the first compound in an amount of from 1 wt. % to 15 wt. %:

8. The organic electronic element according to claim 7, wherein the first compound is a p-type dopant.

9. The organic electronic element according to claim 1, wherein the first electrode is an anode electrode, and the second electrode is a cathode electrode.

10. The organic electronic element according to claim 9, wherein the first electrode is a transparent electrode, and the second electrode is a semitransparent reflective electrode.

11. The organic electronic element according to claim 1, further comprising a capping layer on the second electrode.

12. The organic electronic element according to claim 1, wherein the first light emitting layer and the second light emitting layer are a blue light emitting layer.

13. The organic electronic element according to claim 1, wherein the second light emitting layer is disposed to have a distance within a range of from 200 Å to 800 Å from a surface of the second electrode.

14. The organic electronic element according to claim 1, wherein the second light emitting layer is disposed to have a distance within a range of from 1000 Å to 1600 Å from a surface of the second electrode.

15. The organic electronic element according to claim 1, wherein the first light emitting layer is disposed to have a distance within a range of from 1800 Å to 2400 Å from a surface of the second electrode.

16. The organic electronic element according to claim 1, wherein the first light emitting layer is disposed to have a distance within a range of from 2600 Å to 3200 Å from a surface of the second electrode.

17. A display panel comprising a subpixel comprising the organic electronic element of claim 1.

18. A display device comprising:
the display panel of claim 17; and
a driving circuit for driving the display panel.
